(19) United States
(12) Patent Application Publication
(10) Pub. No.: US 2007/0068071 A1
(43) Pub. Date: Mar. 29, 2007

(54) Compositions and methods for hydrogen generation

(76) Inventors: Michael T. Kelly, Plainsboro, NJ (US); Kevin W. McNamara, Red Bank, NJ (US); Xiaolei Sun, Chandler, AZ (US)

Correspondence Address: DICKSTEIN SHAPIRO LLP, 1825 EYE STREET NW, Washington, DC, 20006-5403, US

(21) Appl. No.: 11/524,446

(22) Filed: Sep. 21, 2006

Related U.S. Application Data
(60) Provisional application No. 60/718,748, filed on Sep. 21, 2005.
(60) Provisional application No. 60/718,749, filed on Sep. 21, 2005.
(60) Provisional application No. 60/748,598, filed on Dec. 9, 2005.
(60) Provisional application No. 60/748,599, filed on Dec. 9, 2005.

(51) Int. Cl. C07C 11/24

(52) U.S. Cl. 44/905; 48/1

(57) ABSTRACT

Hydrogen storage fuel compositions comprising a mixture of at least one chemical hydride compound and at least one compound, polymer, or salt that acts as a water surrogate source, and methods for thermally initiated hydrogen generation from fuel compositions, are disclosed. The water surrogate source/chemical hydride compositions are preferably solids, and may be powders, caplets, tablets, pellets or granules, for example. The water surrogate source/chemical hydride compositions may comprise alternating layers of the chemical hydride and of the water surrogate source.

This application claims the benefit of U.S. Provisional Application Ser. No. 60/718,748, filed Sep. 21, 2005, of U.S. Provisional Application Ser. No. 60/718,749, filed Sep. 21, 2005, of U.S. Provisional Application Ser. No. 60/748,598, filed Dec. 9, 2005, and of U.S. Provisional Application Ser. No. 60/748,599, filed Dec. 9, 2005, the entire disclosures of all of which are incorporated herein by reference.

### FIELD OF THE INVENTION

The present invention relates to hydrogen storage fuel compositions comprising a mixture of at least one chemical hydride compound and at least one compound, polymer, or salt that acts as a water surrogate source. The invention also relates to methods for thermally initiated hydrogen generation from fuel compositions.

### BACKGROUND OF THE INVENTIONS

Hydrogen is the fuel of choice for fuel cells; however, its widespread use is complicated by the difficulties in storing the gas. Various nongaseous hydrogen carriers, including hydrocarbons, metal hydrides, and chemical hydrides are being considered as hydrogen storage and supply systems. In each case, systems need to be developed in order to release the hydrogen from its carrier, either by reformation as in the case of hydrocarbons, desorption from metal hydrides, or catalyzed hydrolysis of chemical hydrides.

### DETAILED DESCRIPTION OF THE INVENTION

The invention provides hydrogen storage compositions comprising at least one chemical hydride and at least one water surrogate source, and heat-activated methods of hydrogen generation in which hydrogen is generated by the hydrolysis reaction of a chemical hydride, which reaction may be initiated by the application of heat to a mixture comprising at least one chemical hydride compound and at least one water surrogate source. Chemical hydride compounds undergo a reaction with the water surrogate to generate hydrogen, wherein the stoichiometry is determined by the number of water molecules necessary for oxidation of the chemical hydride compound. The rate of hydrogen generation can be accelerated with the application of heat, or a catalyst, or both.

Water surrogate sources useful in the invention can be characterized as “chemical water” or “bound water,” which terms are defined below. A mixture of water surrogate sources may be used to control activation temperature, pH, reaction conditions, and product properties.

The term “chemical water” as used in the present invention is intended to encompass a compound, polymer, or salt that generates water equivalents via intramolecular or intermolecular reactions that occur upon warming to a temperature preferably above ambient. Chemical water species do not contain molecular water in the form of H₂O molecules. Preferably, the chemical water species releases the water equivalents at a temperature between about 40° C. to 350° C., preferably between about 70° C. to about 250° C., and most preferably above about 100° C.

Chemical water species can release water equivalents via a chemical reaction such as dehydration of a compound containing hydroxyl groups. In this sense, water is not simply water molecules present in the lattice of a salt, but the water equivalent is a product of a thermally initiated chemical reaction. Classes of compounds which undergo such a thermal dehydration include, for example, carbohydrates, borates, and allylic alcohols. It is important to note that, although some compounds and classes (for example, carbohydrates, sodium metaborate dihydrate, and magnesium borate trihydrate) contain the term “hydrates” in their name, they do not necessarily contain free water in the form of H₂O molecules within their structures. Rather, the water equivalent is present as hydrogen and oxygen atoms, for example, in hydroxyl and hydrogen groups, within such compounds or salts.

The term “bound water” as used in the present invention is intended to encompass water molecules contained within the structure of a compound, polymer, or salt, that can be released upon warming to a temperature above ambient. The water may be released from the bound water species by various processes, including, but not limited to, melting, decomposition, or polymorph conversion. Preferably, the bound water species releases the water at a temperature between about 40° C. to 350° C., preferably between about 40° C. and 250° C., preferably above about 100° C.

Bound water species include hydrated salts wherein water molecules are contained in the crystal lattice of a salt. At room temperature the hydrated salt is in the solid state, but at elevated temperature “free” water is available for reaction. Many hydrated salts are polyhydrates and will lose sequential water molecules at different temperatures. For example, a first water of hydration can be lost at a temperature T₁, a second water of hydration can be lost at a second temperature, T₂. Bound water species further include gelled water stored in a hydrated polymer, some of which can store significant amounts of water. For example, hydrated polyacrylate has the capability to store a large amount of water, up to 300 times its own weight.

Suitable chemical hydrides include, but are not limited to, boron hydrides, ionic hydride salts, and aluminum hydrides. Suitable boron hydrides include, without intended limitation, the group of borohydride salts [M(BH₄)ₙ], triborohydride salts [M (B₃H₈)ₙ], decahydrodecaborate salts [M₂(B₁₀H₁₀)ₙ], tridecahydrodecaborate salts [M(B₁₀H₁₃)ₙ], dodecahydrododecaborate salts [M₂(B₁₂H₁₂)ₙ], and octadecahydroicosaborate salts [M₂(B₂₀H₁₈)ₙ], where M is an alkali metal cation, alkaline earth metal cation, aluminum cation, zinc cation, ammonium cation, or phosphonium cation, and n is equal to the charge of the cation; and neutral borane compounds, such as decaborane(14) (B₁₀H₁₄), ammonia borane compounds of formula NHₓBH_{y}, wherein x and y independently=1 to 4 and do not have to be the same, and NHₓRBH_{y}, wherein x and y independently=1 to 4 and do not have to be the same, and R is a methyl or ethyl group. These chemical hydrides may be utilized in mixtures or individually. As used herein, the term “ammonium cation” includes unsubstituted (e.g., NH4+) and alkyl substituted (e.g., mono-, di-, tri-, or tetra-alkyl) ammonium cations.

Ionic hydrides included, without intended limitation, the hydrides of alkali metals and alkaline earth metals such as lithium hydride, sodium hydride, magnesium hydride, and calcium hydride. Aluminum hydrides include, such as, alane (AlH₃) and aluminum hydride salts including, without intended limitation, salts with the general formula M(AlH₄)ₙ, where M is an alkali metal cation, alkaline earth metal cation, aluminum cation, zinc cation, or ammonium cation, and n is equal to the charge of the cation.

An optional component of any water surrogate source/chemical hydride system as described above is a metal salt, where the metal salt can be reduced to a hydrolysis catalyst on exposure to a chemical hydride, such as, for example, a borohydride. Preferably, for the greatest storage efficiency, the catalyst precursor is also a hydrated salt, though non-hydrated salts are suitable. Nonlimiting examples of metal salts include the chloride salts of cobalt, nickel, ruthenium, rhodium, platinum, and copper. Examples of hydrated salts include, without intended limitation, CoCl₂.6H₂O, NiCl₂.6H₂O, RuCl₃.HO, RhCl₃.H₂O, PtCl₄.5H₂O, and CuCl₂.H₂O. The borohydride or other suitable chemical hydride will react with the transition metal salt to form a metal-based hydrolysis catalyst. The catalyst helps promote the reaction even in a basic environment.

Optionally, the boron or other chemical hydride fuel component may be combined with a stabilizer agent. Stabilized fuel compositions comprising borohydride and hydroxide salts are disclosed in co-pending U.S. patent application Ser. No. 11/068,838 entitled “Borohydride Fuel Composition and Methods” and filed on Mar. 2, 2005, which is incorporated by reference herein in its entirety.

The water surrogate source/chemical hydride compositions are preferably solids, and may be powders, caplets, tablets, pellets or granules, for example. In some embodiments, the water surrogate source/chemical hydride compositions are liquid or gelatinous. For example, the admixture of certain hydrated salts and chemical hydride compounds may produce a eutectic, e.g., a mixture of two or more components which has a lower melting point than any of its constituents, and the resultant mixture is in the liquid state at ambient temperature. The individual components may be physically mixed together and/or held in close contact, the water surrogate source and chemical hydride components may be combined into a pellet, caplet, gel, or tablet comprising at least two components, or the water surrogate source and chemical hydride may be held in close contact as separate layers in, for example, a composite. The water surrogate source and chemical hydride are combined in proportions ranging from a 4-fold molar excess of the stoichiometric ratio of water equivalent to a 4-fold excess of the stoichiometric ratio of chemical hydride base on the stoichiometric ratio required by the hydrolysis reaction. Preferably, the water surrogate source and chemical hydride are combined in proportions equivalent to the stoichiometric ratio of water equivalent to chemical hydride required by the hydrolysis reaction. As used herein, molar water equivalent means the number of moles of water a water surrogate source provides.

Hydrogen is generated from the water surrogate source/chemical hydride mixtures when heat is applied to the mixture. Heating elements suitable for use in the invention include, but are not limited to, resistance heaters, nickel-chromium resistance wires, and heat exchangers. The heating can be achieved, for example, by placing the materials in a reactor and heating the reactor, or by a heating element in contact with the water surrogate source/chemical hydride mixture.

In one embodiment of the invention, fuel compositions comprise a mixture of at least one carbohydrate with at least one chemical hydride. For the purposes of determining stoichiometry, the carbohydrates can be written as, for example, Cₓ(H₂O)_{y} compounds or CₓH₂(H₂O)_{y} compounds where x and y are integers; other stoichiometric ratios can be determined using the teachings herein. For example, one mole of C₆H₁₂O₆ has a molar water equivalent of 6 when written as C₆(H₂O)₆, wherein the water may be obtained by a dehydration mechanism such as that illustrated in Equation (1) for a monosaccharide. The disaccharides and polysaccharides undergo dehydration of the hydroxyl groups in a similar fashion.
C₆H₁₂O₆→6C+6H₂O (1)

The class of carbohydrates includes monosaccharides such as the hexoses (e.g., glucose, fructose, mannose, and galactose), the pentoses (e.g., ribose, and xylose), the tetroses (e.g. erythrose); disaccharides such as sucrose; polysaccharides such as starch, cellulose, and glycogen; and sugar alcohols such as mannitol, sorbitol, xylitol, inositol, and glycerol. In the carbohydrate case, both aldoses and ketoses are suitable.

In another embodiment of the present invention, fuel compositions according to the present invention may comprise mixtures of at least one chemical hydride with at least one hydrated salt. Suitable hydrated salts include, without intended limitation, borates, chlorides, phosphates, carbonates, bisulfates, and sulfates, wherein the cation is an alkali metal ion, an alkaline earth metal ion, zinc ion, aluminum ion, or ammonium ion, or a combination thereof as in a double salt. Representative examples of hydrated salts include, without intended limitation, magnesium sulfate heptahydrate (MgSO₄.7H₂O, a molar water equivalent of 7), magnesium chloride hexahydrate (MgCl₂.6H₂O, a molar water equivalent of 6), trisodium phosphate (Na₃PO₄.12H₂O, a molar water equivalent of 12), calcium sulfate dihydrate (CaSO₄.2H₂O, a molar water equivalent of 2), sodium carbonate decahydrate (Na₂CO₃.10H₂O), aluminum sulfate octadecahydrate (Al₂(SO₄)₃.18H₂O), sodium aluminum sulfate (NaAl(SO₄)₂.12H₂O, a molar water equivalent of 12), and potassium aluminum sulfate (KAl(SO₄)₂.12H₂O); the molar water equivalent of such salts is determined by the moles of water contained within a mole of hydrate. Salts can be chosen for use in fuel compositions according to the teachings herein by consideration of the weight fraction of water in the hydrated solid, the temperature of water-release, the presence of a cation and anion that cannot be reduced by the chemical hydride, and molecular weight.

In a further embodiment off the present invention, fuel compositions may comprise mixtures of at least one chemical hydride with at least one borate salt. Many of the borate salts contain a portion of their water as waters of hydration and a portion as hydroxyl groups, and can be written in the format jM₂Oₙ.kB₂O₃.XH₂O, wherein n is equal to the charge of the metal cation M. The molar water equivalent of such salts is represented by XH₂O.

Representative examples of borates useful as water surrogates in the embodiments described above, include, without intended limitation, sodium tetraborate decahydrate (Na₂B₄O₇.10H₂O, Na₂O.2B₂O₃.10H₂O); sodium tetraborate pentahydrate (Na₂B₄O₇.5H₂O, or Na₂O.2B₂O₃.5H₂O); sodium metaborate tetrahydrate (NaBO₂.4H₂O, or ½Na₂O.½B₂O₃.4H₂O), disodium tetraborate tetrahydrate (Na₂B₄O₇.4H₂O, or Na₂O.2B₂O₃.4H₂O), ulexite (NaCaB₅O₉.8H₂O, or ½Na₂O.CaO. 5/2B₂O₃.8H₂O), probertite (NaCaB₅O₉.5H₂O, or ½Na₂O.CaO. 5/2B₂O₃.5H₂O), lithium pentaborate pentahydrate (LiB₅O₈.5H₂O, or ½Li₂O. 5/2B₂O₃.5H₂O), potassium tetraborate tetrahydrate (K₂B₄O₇.4H₂O, or K₂O.2B₂O₃.4H₂O); ammonium pentaborate octahydrate (NH₄B₅O₈.4H₂O, or (NH₄)₂O.5B₂O₃.8H₂O), zinc borate heptahydrate (2ZnO.3B₂O₃.7 to 7.5H₂O), zinc borate nonahydate (2 ZnO.3B₂O₃.9H₂O), sodium metaborate dihydrate (NaB(OH)₄, or ½Na₂O.½B₂O₃.2H₂O), sodium pentaborate pentahydrate (NaB₅O₈.5H₂O, or ½Na₂O. 5/2B₂O₃.5H₂O), and pinnoite (MgB₂O₄.3H₂O, or MgO.B₂O₃.3H₂O). Disodium tetraborate tetrahydrate (Na₂B₄O₇.4H₂O, or Na₂O.2B₂O₃.4H₂O), sodium metaborate dihydrate (NaB(OH)₄, or ½Na₂O.½B₂O₃.2H₂O), and pinnoite (MgB₂O₄.3H₂O, or MgO.B₂O₃.3H₂O) do not contain any wafers of hydration; the application of heat to such compounds produces a water equivalent and a boron oxide compound.

In another embodiment, fuel compositions according to the present invention may comprise mixtures of at least one chemical hydride with gelled water stored in a hydrated polymer such as polyacrylic acid [PAA], polyacrylamide, poly(2-hydroxyethyl methacrylate) [poly-HEMA], poly(iso-butylene-co-maleic acid), poly(acrylic acid-co-acrylamide). The cation and degree of cross-linking of the polymer can be varied to change the water uptake properties and alter physical and chemical characteristics such as viscosity. The molar water equivalents of such hydrated polymers are determined by the amount of water carried by the gel. We have demonstrated that it is possible to make a stable gel by mixing poly-HEMA powder with an aqueous alkaline sodium borohydride solution to produce a gel that produces hydrogen when heat is applied.

In another embodiment of the present invention, fuel compositions may comprise mixtures of at least one chemical hydride with at least one bicarbonate salt, wherein the cation is an alkali metal ion, an alkaline earth metal ion, zinc ion, aluminum ion, or ammonium ion. The bicarbonate salts contain hydroxyl groups that can be converted to oxides and water. For example, sodium bicarbonate is converted to sodium carbonate, carbon dioxide, and water at temperatures between about 50 to about 100° C., as illustrated in Equation (2).
2NaHCO₃→Na₂CO₃+H₂O+CO₂ (2)

The following examples further describe and demonstrate features of the present invention. The examples are given solely for the purpose of illustration and are not to be construed as a limitation of the present invention.

### EXAMPLE 1

A mixture of sodium borohydride and D-fructose (C₆H₁₂O₆, 6 molar water equivalent) was combined in a ratio of 1 mole sodium borohydride to 1 mole of fructose (alternatively described as a ratio of 1 mole of sodium borohydride to 6 molar water equivalents), and loaded into a Parr autoclave reactor. The reaction temperature was stepped from room temperature to about 70° C. and then to about 250° C. Hydrogen generation was initiated at about 70° C., with complete conversion of borohydride to hydrogen at about 250° C. The amount of hydrogen generated was equivalent to 3.7 wt-% of the reactants' weight.

### EXAMPLE 2

A mixture of sodium borohydride and magnesium chloride hexahydrate (MgCl₂.6H₂O, 6 molar water equivalent) was loaded into a Parr autoclave reactor. As the reactor temperature increased to about 110° C., limited hydrogen gas pressure in the reactor was observed with about 21% borohydride conversion to hydrogen. Borohydride conversion increased from about 21% to about 74%, as the reactor temperature was increased to about 150° C. in about 100 minutes. The amount of hydrogen generated was equivalent to 4.6% of the reactants' weight.

### EXAMPLE 3

A mixture of sodium borohydride and borax decahydrate (Na₂B₄O₇.10H₂O, 10 molar water equivalent) in a ratio of 2 moles sodium borohydride to 1 mole borax decahydrate (or, alternatively described as a ratio of 2 moles of sodium borohydride to 10 molar water equivalents) was loaded into a cylindrical glass reactor with 2 wt-% CoCl₂.6H₂O catalyst. The reaction was carried out in a semi-batch mode. The generated hydrogen was measured through a mass flow meter. The reactor was heated by using an oil bath. Hydrolysis of borohydride was initiated at 70° C., and the hydrogen generation rate-reached 600 standard cubic centimeters (sccm). The amount of hydrogen generated was equivalent to a hydrogen storage density of 3.5 wt-% of the combined weight of reactants.

### EXAMPLE 4

At room temperature, poly(2-hydroxyethyl methacrylate) [poly(2-HEMA)] (Mv=1,000,000) was added in 3:1, ratio by weight to a fuel solution comprising 20 wt-% sodium borohydride and 3 wt-% sodium hydroxide. After the addition of poly(2-HEMA), the liquid fuel turned into gel. Thermogravimetric analysis (TGA) of the poly(2-HEMA)/fuel mixture indicated that water was released from the gel at elevated temperature. With a 10° C./min heating rate starting from room temperature, hydrolysis of sodium borohydride and hydrogen generation was observed at a temperature of about 150° C.

### EXAMPLE 5

A mixture of lithium hydride and D-fructose (C₆H₁₂O₆, 6 molar water equivalent) was combined in a ratio of 12 moles of lithium hydride to 1 mole of fructose and loaded into a Parr autoclave reactor. The reaction temperature was stepped every 50° from room temperature, to about 300° C. Hydrogen generation was initiated at about 70° C. with about 50% of the hydride converted to hydrogen. The amount of hydrogen generated was equivalent to 4.0% of the reactants' weight.

While the present invention has been described with respect to particular disclosed embodiments, it should be understood that numerous other embodiments are within the scope of the present invention. It is not intended that the present invention be limited to the illustrated embodiments. Any modification of the present invention that comes within the spirit and scope of the following claims should be considered part of the present invention.

What is claimed as new and desired to be protected by Letters Patent of the United States is:

1. A fuel composition comprising at least one chemical hydride and at least one water surrogate, source comprising a material selected from the group consisting of carbohydrates, allylic alcohols, polymers, hydrated salts and bicarbonate salts.

2. The fuel composition of claim 1, wherein the at least one chemical hydride is selected from the group consisting of boron hydrides, ionic hydride salts, and aluminum hydrides.

3. The fuel composition of claim 2, wherein the chemical hydride is a boron hydride selected from the group consisting of borohydride salts [M(BH₄)ₙ], triborohydride salts [M(B₃H₈)ₙ], decahydrodecaborate salts [M₂(B₁₀H₁₀)ₙ], tridecahydrodecaborate salts [M(B₁₀H₁₃)ₙ], dodecahydrododecaborate salts [M₂(B₁₂H₁₂)ₙ], and octadecahydroicosaborate salts [M₂(B₂₀H₁₈)ₙ] where M is an alkali metal cation, alkaline earth metal cation, aluminum cation, zinc cation, ammonium cation, alkyl ammonium cation, dialkyl ammonium cation, triakly ammonium cation, tetraalkyl ammonium cation, or phosphonium cation, and n is equal to the charge of the cation.

4. The fuel composition of claim 2 further comprising a stabilizer.

5. The fuel composition of claim 2, wherein the chemical hydride comprises a borane compound.

6. The fuel composition of claim 5, wherein the borane compound is selected from the group consisting of decaborane(14) (B₁₀H₁₄), ammonia borane compounds of formula NHₓBH_{y}, and NHₓRBH_{y}, wherein x and y are independently selected from 1 to 4, and wherein R is a methyl or ethyl group.

7. The fuel composition of claim 2, wherein the chemical hydride is a hydride salt selected from the group consisting of hydrides of alkali metals and alkaline earth metals.

8. The fuel composition of claim 2, wherein the chemical hydride is an aluminum hydride selected from the group consisting of alane (AlH₃) and aluminum hydride salts.

9. The fuel composition of claim 8, wherein the aluminum hydride salts have the formula M(AlH₄)ₙ, where M is an alkali metal cation, alkaline earth metal cation, aluminum cation, zinc cation, or ammonium cation, and n is equal to the charge of the cation.

10. The fuel composition of claim 1, wherein the water surrogate source comprises a carbohydrate selected from the group consisting of monosaccharides, disaccharides, polysaccharides and sugar alcohols.

11. The fuel composition of claim 1, wherein the water surrogate source comprises a polymer selected from the group consisting of polyacrylic acid [PAA], polyacrylamide, poly(2-hydroxyethyl methacrylate) [poly-HEMA], poly(iso-butylene-co-maleic acid) and poly(acrylic acid-co-acrylamide).

12. The fuel composition of claim 1, wherein the water surrogate source comprises a hydrated salt selected from the group consisting of chlorides, phosphates, carbonates, bisulfates, and sulfates.

13. The fuel composition of claim 12, wherein the hydrated salt is selected from the group consisting of magnesium sulfate heptahydrate (MgSO₄.7H₂O), magnesium chloride hexahydrate (MgCl₂.6H₂O), trisodium phosphate (Na₃PO₄.12H₂O), calcium sulfate dihydrate (CaSO₄.2H₂O), sodium carbonate decahydrate (Na₂CO₃.10H₂O), aluminum sulfate octadecahydrate (Al₂(SO₄)₃.18H₂O), sodium aluminum sulfate (NaAl(SO₄)₂.12H₂O, a molar water equivalent of 12), and potassium aluminum sulfate (KAl(SO₄)₂.12H₂O).

14. The fuel composition of claim 1, wherein the composition is in a form selected from the group consisting of granules, pellets, gel, tablets or powder, or a combination thereof.

15. The fuel composition of claim 1, wherein the composition is in the form of a composite comprising at least one layer of the at least one chemical hydride and at least one layer of the water surrogate source.

16. The fuel composition of claim 1, further comprising a hydrolysis catalyst.

17. The fuel composition of claim 16, wherein the hydrolysis catalyst comprises a material selected from the group consisting of chloride salts of cobalt, chloride salts of nickel, chloride salts of ruthenium, chloride salts of rhodium, chloride salts of platinum, and chloride salts of copper.

18. A fuel composition comprising at least one chemical hydride and at least one carbohydrate water surrogate.

19. The fuel composition of claim 18, wherein the at least one chemical hydride is selected from the group consisting of boron hydrides, ionic hydride salts, and aluminum hydrides.

20. The fuel composition of claim 18 further comprising a stabilizer.

21. The fuel composition of claim 18, wherein the at least one carbohydrate water surrogate is selected from the group consisting of monosaccharides, disaccharides, polysaccharides and sugar alcohols.

22. The fuel composition of claim 18, wherein the at least one carbohydrate water surrogate is an aldose or a ketose.

23. The fuel composition of claim 18, wherein the at least one carbohydrate water surrogate is selected from the group consisting of glucose, fructose, xylose, mannitol, sorbitol, xylitol, and glycerol.

24. The fuel composition of claim 18, wherein the at least one chemical hydride is sodium borohydride.

25. The fuel composition of claim 18, wherein the at least one chemical hydride is lithium hydride.

26. The fuel composition of claim 18, wherein the composition is a mixture in a form selected from the group consisting of granules, pellets, gel, tablets or powder, or a combination thereof.

27. The fuel composition of claim 18, wherein the composition is a composite comprising at least one layer of the at least one chemical hydride and at least one layer of the carbohydrate water surrogate.

28. A fuel composition, comprising at least one chemical hydride and at least one hydrated polymer.

29. The fuel composition of claim 28, wherein the at least one chemical hydride is selected from the group consisting of boron hydrides, ionic hydride salts, and aluminum hydrides.

30. The fuel composition of claim 28 further comprising a stabilizer.

31. The fuel composition of claim 28, wherein the at least one hydrated polymer comprises a material selected from the group consisting of polyacrylic acid [PAA], polyacrylamide, poly(2-hydroxyethyl methacrylate) [poly-HEMA], poly(iso-butylene-co-maleic acid) and poly(acrylic acid-co-acrylamide).

32. The fuel composition of claim 28, wherein the at least one chemical hydride is sodium borohydride.

33. The fuel composition of claim 28, wherein the composition is a mixture in a form selected from the group consisting of granules, pellets, gel, tablets or powder, or a combination thereof.

34. The fuel composition of claim 28, wherein the composition is a composite comprising at least one layer of the at least one chemical hydride and at least one layer of the hydrated polymer.

35. A fuel composition, comprising at least one chemical hydride and at least one water surrogate selected from the group consisting of hydrated salts of chlorides, phosphates, carbonates, bisulfates and sulfates.

36. The fuel composition of claim 35, wherein the hydrated salt is selected from the group consisting of magnesium sulfate heptahydrate (MgSO₄.7H₂O), magnesium chloride hexahydrate (MgCl₂.6H₂O), trisodium phosphate (Na₃PO₄.12H₂O), calcium sulfate dihydrate (CaSO₄.2H₂O), sodium carbonate decahydrate (Na₂CO₃.10H₂O), aluminum sulfate octadecahydrate (Al₂(SO₄)₃.18H₂O), sodium aluminum sulfate (NaAl(SO₄)₂.12H₂O, a molar water equivalent of 12), and potassium aluminum sulfate (KAl(SO₄)₂.12H₂O).

37. The fuel composition of claim 35, wherein water in the hydrated salt comprises molecular water contained in the lattice of the salt.

38. The fuel composition of claim 35, wherein the at least one chemical hydride is selected from the group consisting of boron hydrides, ionic hydride salts, and aluminum hydrides.

39. The fuel composition of claim 35 further comprising a stabilizer.

40. The fuel composition of claim 35, wherein the at least one chemical hydride is sodium borohydride.

41. The fuel composition of claim 35, wherein the composition is a mixture in a form selected from the group consisting of granules, pellets, gel, tablets or powder, or a combination thereof.

42. A fuel composition, comprising a mixture of at least one chemical hydride and at least one borate salt, wherein at least a portion of the water contained in the borate salt is in a hydroxyl group configuration.

43. The fuel composition of claim 42, wherein the at least one chemical hydride is selected from the group consisting of boron hydrides, ionic hydride salts, and aluminum hydrides.

44. The fuel composition of claim 42 further comprising a stabilizer.

45. The fuel composition of claim 42, wherein all water contained in the borate salt is in hydroxyl group configuration.

46. The fuel composition of claim 42, wherein the borate salt is selected from the group consisting of disodium tetraborate tetrahydrate, sodium metaborate dihydrate, and pinnoite.

47. A fuel composition, comprising a mixture of at least one chemical hydride and at least one water surrogate source which does not comprise sodium borate or lithium borate.

48. The fuel composition of claim 47, wherein the chemical hydride is selected from the group consisting of boron hydrides, ionic hydride salts, and aluminum hydrides.

49. The fuel composition of claim 47 further comprising a stabilizer.

50. The fuel composition of claim 47, wherein the chemical hydride comprises at least one of sodium borohydride and lithium borohydride.

51. The fuel composition of claim 47, wherein the water surrogate source is selected from the group consisting of carbohydrates, allylic alcohols, polymers, hydrated salts, bicarbonate salts, and borate salts.

52. A fuel composition, comprising a mixture of at least one chemical hydride selected from the group consisting of sodium borohydride and lithium borohydride, and a water surrogate source which does not comprise sodium borate or lithium borate in a form providing water only as bound molecular water.

53. The fuel composition of claim 52, wherein the water surrogate source comprises a material selected from the group consisting of polymers, hydrated salts and borate salts.

54. A fuel composition, comprising a mixture of at least one chemical hydride and at least one water surrogate source comprising a borate salt.

55. The fuel composition of claim 54, wherein the borate salt is selected from the group consisting of sodium tetraborate decahydrate, sodium tetraborate pentahydrate, sodium metaborate tetrahydrate, disodium tetraborate tetrahydrate, ulexite, probertite, lithium pentaborate pentahydrate, potassium tetraborate tetrahydrate, ammonium pentaborate octahydrate, zinc borate heptahydrate, zinc borate nonahydate, sodium metaborate dihydrate, sodium pentaborate pentahydrate, and pinnoite.

56. A fuel composition, comprising:
at least one chemical hydride;
at least one water surrogate source comprising a material selected from the group consisting of carbohydrates, allylic alcohols, polymers, hydrated salts and borate salts, wherein at least one of the borate salts contains at least one portion of its water in a hydroxyl group format; and
a hydrolysis catalyst.

57. The fuel composition of claim 56, wherein the hydrolysis catalyst is a metal salt.

58. The fuel composition of claim 57, wherein the metal salt is selected from the group consisting of chloride salts of cobalt, chloride salts of nickel, chloride salts of ruthenium, chloride salts of rhodium, chloride salts of platinum, and chloride salts of copper.

59. A process for generating hydrogen, comprising:
providing a mixture of at least one chemical hydride and at least one material which is a water surrogate source; and
heating the mixture to release water equivalents via a chemical reaction involving at least one hydroxyl group in the water surrogate to generate hydrogen.

60. The process of claim 59, wherein the water surrogate source comprises a material selected from the group consisting of carbohydrates, allylic alcohols, and borate salts.

61. The process of claim 59, further comprising heating the mixture to a temperature of between about 40 to about 350° C.

62. The process of claim 61 further comprising heating the mixture to a temperature above about 100° C.

63. A process for generating hydrogen, comprising:
providing a mixture of at least one chemical hydride and at least one water surrogate source that comprises a material selected from the group consisting of carbohydrates, allylic alcohols, polymers, hydrated salts and borate salts, wherein at least a portion of the water contained in the borate salts is provided in a hydroxyl group configuration; and
heating the mixture to generate hydrogen.

64. A process for generating hydrogen, comprising:
providing a mixture of at least one chemical hydride and at least one compound which is a water surrogate source and which does not comprise sodium borate or lithium borate; and
heating the mixture to generate hydrogen.

65. The process of claim 64, wherein the mixture of at least one chemical hydride comprises sodium borohydride, lithium borohydride, lithium hydride, or combinations thereof.
